(19) United States
(12) Patent Application Publication
(10) Pub. No.: US 2026/0250708 A1
(43) Pub. Date: Aug. 27, 2026

(54) METHODS FOR ASSESSING RESISTANCE TO NON-INTEGRATING VIRUS VECTORS

(71) Applicant: UNIVERSITE DE NAMUR, Namur (BE)

(72) Inventors: Jonathan DOUXFILS, Hingeon (BE); Constant GILLOT, Wierde (BE); Vincent MALOTEAU, Presles (BE); Julien FAVRESSE, Watermael-Boitsfort (BE)

(73) Assignee: UNIVERSITE DE NAMUR, Namur (BE)

(21) Appl. No.: 18/726,876

(22) PCT Filed: Jan. 6, 2023
(86) PCT No.: PCT/EP2023/050215

§ 371 Date: Jul. 5, 2024

(30) Foreign Application Priority Data
Jan. 7, 2022 (EP) 22150499.6

(51) Int. Cl. C12N 15/86; A61K 39/00; A61K 39/215; A61P 37/04; C07K 14/005; G01N 33/50

(52) U.S. Cl. CPC C12N 15/86; A61K 39/215; A61P 37/04; C07K 14/005; G01N 33/5023; A61K 2039/5256; C12N 2710/10043; C12N 2770/20022; C12N 2770/20034; G01N 2333/165

(57) ABSTRACT

An in vitro method for assessing the ability of a subject to produce a therapeutic or prophylactic molecule encoded by a non-integrative viral vector. The method is also useful for assessing a subject's resistance towards a non-integrating virus vector. Also, a kit for implementing the method, which includes an element for determining or measuring the therapeutic or prophylactic molecule encoded by a non-integrative viral vector.

### FIELD OF INVENTION

The present invention relates to an in vitro method for assessing the efficacy of a non-integrative virus vector-based vaccine or gene therapy in a subject. The present invention also relates to the detection of a resistance to non-integrating virus vectors within the serum of subjects, in particular pre-existing immune resistance.

### BACKGROUND OF INVENTION

The adenovirus vaccine known as ChadOx1 nCoV19 and produced by the company AstraZeneca under the brand name of Vaxzevria® is one of the main vaccines in the fight against COVID-19 pandemic. The use of adenoviruses to develop vaccination strategies has been known for almost 20 years. The adenovirus used in this vaccine is a chimpanzee adenovirus (ChAdOx1 nCoV-19) modified to contain a fragment of the SARS-CoV-2 genetic material.

This fragment of genome permits the cells infected by this adenovirus to synthetize spike protein (S) and to secrete it outside the cells. This S protein will then be detected by the antigen presenting cells of the immune system, allowing the production of antibodies directed against the S protein after the recruitment of T-helper cells and the subsequent stimulation of B lymphocytes. The S protein is responsible for the adhesion and the fusion of SARS-CoV-2 with the host cell. Therefore, anti-S antibodies have the potential to neutralize the entry of the virus into human cells.

Regarding the viral vector, i.e., the adenovirus, only the cells that are capable of interacting with this adenovirus are potentially able to integrate the Spike-encoding RNA of SARS-CoV-2 into their cytoplasm and should therefore present the characteristics to interact with the viral vector. These are cells expressing the coxsackievirus and adenovirus receptor (CAR) at their surface (FEBS Lett. 2020; 594(12):1828-37; Virology. 2017; 502:144-51).

Adenoviruses are well known as vectors for potential gene therapies or as vaccine vectors, as well as other non-integrating virus such as adeno-associated viruses (AAV), but in some cases, there is a potential pre-existing immunity that reduces the usefulness of some of these adenoviruses and AAV when used as viral vector. What generates this immunity is still unclear; the cytosolic receptor TRIM21 could be involved via a mechanism that is still not well understood (PNAS, 2018; 115(41):10440-5).

There is therefore a need to provide a solution for detecting if a patient will be responsive, or not, to a prophylactic or therapeutic approach relying on adenoviral vectors or other non-integrative viral vectors, such as adenoviral vector-based vaccines.

Nwanegbo et al. (Clinical and diagnostic laboratory immunology vol. 11, 2 (2004): 351-7) previously showed in vitro detection of pre-existing resistance against adenovirus serotypes 5 and 35 in the serum of individuals, comprising detecting the fluorescence of EGFP and/or EYFP in cells infected by recombinant adenoviruses encoding these fluorescent proteins. However, the work of Nwanegbo et al. does not investigate the effect of this resistance on the ability of an adenoviral vector (and not a recombinant adenovirus) to induce the secretion of a therapeutic molecule in a patient. In other words, the work of Nwanegbo et al. focused on the detection of infected cells per se, but cannot be extrapolated as to predict the efficacity of an adenoviral vector-based vaccine, i.e., the ability to secrete a protein of interest after infection.

Here, the inventors provide a solution for predicting if the molecule encoded by the non-integrative viral vector will be produced and secreted by the cells of the patient, and therefore exerts its prophylactic and/or therapeutic effect. The invention further concerns a method for assessing the resistance towards a non-integrating virus vector and identify the cause of the resistance.

### SUMMARY

The present invention thus relates to an in vitro method for assessing the ability of a subject to produce at least one therapeutic molecule of interest encoded by a non-integrative viral vector, comprising the steps of:
(i) culturing cells expressing an attachment receptor specific of the non-integrating viral vector;
(ii) incubating the cells with a composition comprising the non-integrating viral vector and a serum sample of the subject in a culture medium;
(iii) measuring the expression level of the at least one therapeutic molecule of interest encoded by the non-integrative viral vector,
wherein an expression level of said at least one molecule of interest superior to a reference value means that said subject is able to produce said at least one molecule of interest.

In some embodiments, the ability to produce the at least one therapeutic molecule of interest means that the subject is not resistant towards the non-integrating virus vector.

In some embodiments, the non-integrating virus vector is an adenovirus vector or an adeno-associated virus (AAV) vector.

In some embodiments, the at least one therapeutic molecule of interest produced by said cultured cells is a protein, polypeptide or peptide encoded by at least one nucleic acid within said non-integrating virus vector. In some embodiments, the at least one therapeutic molecule of interest produced by said cultured cells is a protein encoded by at least one nucleic acid within said non-integrating virus vector.

In some embodiments, the expression level of the at least one therapeutic molecule of interest is measured at the protein level. In some embodiments, the expression level of the at least one therapeutic molecule of interest is measured by the detection and/or quantification of said at least one molecule secreted in the cell culture supernatant.

In some embodiments, the at least one therapeutic molecule of interest is a nucleic acid molecule comprised in or produced from said non-integrating virus vector sequence.

In some embodiments, the expression level of the at least one nucleic acid molecule is measured at the RNA level. In some embodiments, the expression level of the at least one nucleic acid molecule is measured by RNA-seq, microarrays, RT-PCR, RT-qPCR, Northern Blot and/or hybridization techniques.

In some embodiments, the reference value is 0. In some embodiments, the reference value is the expression level of said at least one therapeutic molecule in the serum of a subject not incubated with the non-integrative viral vector encoding said at least one therapeutic molecule.

In certain embodiments, the protein is selected from the group comprising or consisting of viral protein Spike (S), Ebola virus glycoprotein (EBOV GP), Hemagglutinin (HA), Glycoprotein 41 and 120 (Gp41, Gp120), Falciparum circumsporozoite surface antigen, hepatitis C virus NS antigen, mycobacterial antigens 85A, 85B, and TB10.4, CHIKV full-length structural polyprotein (Capsid, E3, E2, 6k and E1), RSV-fusion protein, GI.1 and GII.4 antigen protein from norovirus, factor VIII, factor IX and factor XI.

In some embodiments, the subject is resistant when the at least one therapeutic molecule of interest is substantially undetectable, as measured by the appropriate detection technique.

In some embodiments, the resistance is a pre-existing immune resistance to non-integrating virus vectors.

In some embodiments, the non-integrating virus vector is comprised in a vaccine composition or in a pharmaceutical composition. In some embodiments, the pharmaceutical composition is for preventing or treating cancer or a genetic disease.

In some embodiments, the vaccine is for immunization against a pathogen selected from the group comprising or consisting of Coronaviridae, Filoviridae, Orthomyxoviridae, Togaviridae, Pneumoviridae, Caliciviridae, Retroviridae, Flaviviridae, Mycobacteriaceae, and Plasmodium.

In some embodiments, the vaccine is for immunization against a virus selected from the group comprising or consisting of SARS-CoV-2, Ebola virus, Chikungunya, human immunodeficiency virus (HIV), hepatitis C virus (HCV), Zika virus, respiratory syncytial virus (RSV), Norovirus, Influenza virus, preferably SARS-CoV-2.

In some embodiments, the non-integrating virus is an adenovirus, and the adenovirus attachment receptor is selected from the group comprising or consisting of the coxsackievirus and adenovirus receptor (CAR), CD46, CD80, CD86, sialic acid and integrins.

In some embodiments, the cells are the CAR-expressing cells of a cell line selected from the group comprising or consisting of A549, H460, Caco-2, HEK-293. In one embodiment, the cells are the CAR-expressing cells of a cell line A549.

In some embodiments, step (ii) is performed during at least 5 days.

The present invention further relates to a kit for implementing the in vitro method, the kit comprising means for determining or measuring said at least one therapeutic molecule of interest.

Another object of the present invention is an in vitro method to assess the susceptibility of a subject to respond to a treatment with a non-integrative viral vector encoding at least one molecule of interest, comprising the steps of:
(i) culturing cells expressing an attachment receptor specific of the non-integrating viral vector;
(ii) incubating the cells with a composition comprising the non-integrating viral vector and a serum sample of the subject in a culture medium;
(iii) measuring the expression level of the at least one molecule of interest; and
(iv) assessing if the subject is susceptible to respond to the treatment, wherein an expression level of the at least one molecule of interest superior to a reference value means that the subject is susceptible to respond to the treatment.

The present invention further relates to a method for preventing and/or treating a disease with a non-integrative viral vector encoding at least one molecule of interest in a subject, comprising the steps of:
determining if the subject is susceptible to respond to a treatment with the non-integrative viral vector encoding the at least one molecule of interest by:
(i) culturing cells expressing an attachment receptor specific of the non-integrating viral vector;
(ii) incubating the cells with a composition comprising the non-integrating viral vector encoding the at least one molecule of interest, and a serum sample of the subject in a culture medium;
(iii) measuring the expression level of the at least one molecule of interest; and
(iv) determining if the subject is susceptible to respond to a treatment with the non-integrative viral vector, wherein an expression level of the at least one molecule of interest superior to a reference value means that the subject is susceptible to respond,
administering to the subject susceptible to respond a therapeutically effective amount of the non-integrative viral vector encoding at least one molecule of interest.

### Definitions

In the present invention, the following terms have the following meanings:

“About” preceding a figure means plus or less 10% of the value of said figure.

“Adenovirus vector” or “adenoviral vector” refers to a vector construct comprising nucleotide sequences derived from the genome of a virus of the Adenoviridae family and, optionally, one or more heterologous nucleic acid sequence of interest. Said heterologous nucleic acid sequence of interest, once within the host cell, encodes at least one protein or peptide of interest.

“Adeno-associated virus vector” or “adeno-associated viral vector” refers to a vector construct comprising nucleotide sequences derived from the genome of a virus of the Parvoviridae family and, optionally, one or more heterologous nucleic acid sequence of interest. Said heterologous nucleic acid sequence of interest, once within the host cell, encodes at least one protein or peptide of interest.

“And/Or” refers to and encompasses any and all possible combinations of one or more of the associated listed items, as well as the lack of combinations when interpreted in the alternative (“or”).

“Treating” or “treatment” refers to both therapeutic treatment and prophylactic or preventative measures; wherein the object is to prevent or slow down (lessen) the targeted pathologic condition or disorder such as cancer. Those in need of treatment include those already with the disorder as well as those prone to have the disorder or those in whom the disorder is to be prevented. An individual or mammal is successfully “treated” for the disorder if, after receiving a therapeutic amount of an adenovirus vector according to the present invention, the individual shows observable and/or measurable reduction in or absence of one or more of the following: reduction in the number of pathogenic cells; reduction in the percent of total cells that are pathogenic; and/or relief to some extent, one or more of the symptoms associated with the specific disease or disorder such as cancer; reduced morbidity and mortality, and improvement in quality of life issues. The above parameters for assessing successful treatment and improvement in the disease are readily measurable by routine procedures familiar to a physician.

“At least one” includes 1, 2, 3, 4, 5, 6, 7, 8, 9, 10, 25, 50, 75, 100, 250, 500, 750, 10³, 10⁴, 10⁵, 10⁶, 10⁷, 10⁸, 10⁹, 10¹⁰, 10¹¹, 10¹², 10¹³, 10¹⁴, 10¹⁵ or more.

“Comprising”, “comprises” and “comprised of” are used herein are synonymous with “including”, “includes” or “containing”, “contains”, and are inclusive or open-ended and do not exclude additional, non-recited members, elements or method steps. These terms also encompass “consisting of”.

“Immune response” refers to the development in a subject of a cellular and/or antibody-mediated immune response after the administration of a composition or vaccine of interest. The immune response usually includes, but is not limited to, one or more of the following effects: the production of antibodies, B cells, helper T cells, and/or cytotoxic T cells, directed specifically to an antigen or antigens included in the composition or vaccine of interest. Preferably, the subject will display either a therapeutic or protective immune response such that resistance to new infection will be enhanced and/or the clinical severity of the disease reduced.

“Individual” refers to an animal, preferably a mammal, more preferably a human. In one embodiment, the individual is a man. In another embodiment, the individual is a woman. In one embodiment, an individual may be a “subject”, i.e., a warm-blooded animal, more preferably a human, who/which is awaiting the receipt of, or is receiving medical care or was/is/will be the object of a medical procedure. In one embodiment, the individual is an adult (for example a subject above the age of 18). In another embodiment, the individual is a child (for example a subject below the age of 18).

“In vitro method” refers to a method comprising steps performed in vitro (e.g., a measurement of apoptosis) or ex-vivo (e.g., multivariate cox regression model obtained with apoptosis percentage, clinical parameters or biochemical marker previously evaluated on subjects).

“Nucleic acid” or “polynucleotide” refers to any polyribonucleotide or polydeoxyribonucleotide, which may be unmodified RNA or DNA or modified RNA or DNA. “Nucleic acid” or “Polynucleotides” include, without limitation single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically, double-stranded or a mixture of single- and double-stranded regions. In addition, “Nucleic acid” or “polynucleotide” refers to triple-stranded regions comprising RNA or DNA or both RNA and DNA. The term “nucleic acid” or “polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “nucleic acid” or “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Protein”, “polypeptide” and “peptide” are used interchangeably herein to refer to polymers of amino acids of any length. The polymer may be linear or branched, it may comprise modified amino acids, and it may be interrupted by non-amino acids. The terms also encompass an amino acid polymer that has been modified by, for example, disulfide bond formation, glycosylation, lipidation, acetylation, phosphorylation, pegylation, or any other manipulation, such as conjugation with a labelling component. As used herein the term “amino acid” includes natural and/or unnatural or synthetic amino acids, including glycine and both the D or L optical isomers, and amino acid analogs and peptidomimetics.

“Vaccine” refers to any preparation comprising substance or group of substances meant to cause the immune system of a subject to respond to pathogens, such as bacteria or viruses, or to a tumor. Prophylactic vaccines are used to prevent a subject from ever having a particular disease or to only have a mild case of the disease. Such prophylactic vaccines usually comprise the pathogen responsible for the disease, either live and weakened or killed, or components thereof, purified or recombinant. Therapeutic vaccines are intended to treat specific diseases in a subject, in particular cancer. Such therapeutic anti-cancer vaccines comprise a tumor-antigen or tumor-antigens, eliciting an immune response directed against the tumor cells.

### DETAILED DESCRIPTION

Non-integrating virus vectors, such as adenovirus vectors and adeno-associated virus (AAV) vectors, have been used both in anticancer therapy and as vaccines for several years. However, all subjects do not respond equally to these vectors, and some are resistant to them. Consequently, there is an unmet medical need to be able to detect such resistance, in order to avoid potential adverse effect and administer a treatment or vaccine that will be most appropriate to these subjects.

The Applicant herein brings the solution with an in vitro technique enabling the individual detection of a resistance to non-integrating virus vector (see Example 1).

The present invention thus relates to an in vitro method for assessing the ability of a subject to produce at least one molecule of interest encoded by a non-integrative viral vector, comprising the steps of:
(i) culturing cells expressing an attachment receptor specific of the non-integrating viral vector;
(ii) incubating the cells with a composition comprising the non-integrating viral vector and a serum sample of the subject in a culture medium;
(iii) measuring the expression level of the at least one molecule of interest encoded by the non-integrative viral vector;
(iv) comparing the expression level measured in step (iii) with a reference value;
(v) assessing whether the subject is able to produce the at least one molecule of interest, wherein an expression level of the at least one molecule of interest superior to a reference value means that the subject is able to produce the at least one molecule of interest.

As used herein, “non-integrating virus vector” or “non-integrating viral vector” refers to a viral vector whose genetic material remains episomal in the cell cytoplasm. In other words, the genetic material of the virus does not fuse into the host cell genome. In one embodiment, the non-integrating virus vector carries at least one transgene whose expression leads to the production of at least one molecule in the host cell.

In a preferred embodiment, the at least one molecule of interest is a therapeutic molecule of interest.

As used herein, “therapeutic molecule of interest” or “prophylactic molecule of interest” refers to a molecule having at least one beneficial effect on the organism of the subject, i.e., curative and/or prophylactic effect. Typically, the therapeutic molecule of interest or “prophylactic molecule of interest” is for treating at least one disease and/or alleviating or suppressing at least one symptom of a disease, or preventing a disease. Illustratively, the at least one therapeutic molecule of interest may be a protein defective in a genetic disease, an anticancer protein or nucleic acid, or a microbial antigen capable of eliciting an immune response. The nature of the at least one therapeutic molecule of interest will be apparent to the person skilled in the art depending on the disease and/or symptom to be treated. The at least one therapeutic molecule of interest may be present or endogenous, or absent or exogenous from the subject. Within the scope of the present invention, “therapeutic molecule of interest” and “prophylactic molecule of interest” are used interchangeably.

In one embodiment, the at least one molecule of interest is soluble.

In one embodiment, the at least one molecule of interest may be secreted by the cell. In one embodiment, the at least one molecule of interest may be incorporated in an extracellular vesicle or an exosome. In one embodiment, the at least one molecule of interest may be budding at the plasma membrane of the cell.

In some embodiments, the ability of the subject to produce the at least one molecule of interest means that the subject is not resistant towards the non-integrating virus vector. In some embodiments, the ability of the subject to produce the at least one molecule of interest means that the subject is susceptible to be treated by the non-integrating virus vector encoding said at least one molecule of interest.

The determination of whether a subject is able to produce the at least one molecule of interest may vary depending if the at least one molecule of interest is endogenous or exogenous.

The at least one molecule of interest may be exogenous to the subject, i.e., the at least one molecule of interest is not naturally present in, and/or produced by, the subject. According to this embodiment, the natural level of the at least one molecule of interest is equal to 0.

Thus, in some embodiments, the reference value is 0. Accordingly, in some embodiments, an expression level of the at least one molecule of interest superior to 0 means that the subject is able to produce the at least one molecule of interest. In some embodiments, an activity of the at least one molecule of interest superior to 0 means that the subject is able to produce the at least one molecule of interest. In some embodiments, the presence of the at least one molecule of interest means that the subject is able to produce the at least one molecule of interest.

It will be apparent to the person skilled in the art which suitable unit of measurement to use, depending on the nature of the at least one molecule of interest. Illustratively, and non-limitatively, the unit of measurement may be μg/mL, g/L, copies per cell, units per cells, copies per g of tissue, and variants thereof. The unit of measurement may be absolute or relative (e.g., a ratio, a proportion, and the like).

Alternatively, the at least one molecule of interest may be endogenous to the subject, i.e., the at least one molecule of interest is naturally present in, and/or produced by, the subject.

Thus, in some embodiments, the reference value is the expression level of the at least one molecule in the serum of a subject not contacted with the non-integrative viral vector. In some embodiments, an expression level of the at least one molecule of interest increased 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 100-fold, 1,000-fold or more, compared to expression level of the at least one molecule of interest in a subject not contacted with the non-integrative viral vector, means that the subject is able to produce the at least one molecule of interest. In some embodiments, an expression level of the at least one molecule of interest increased 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 100-fold, 1,000-fold or more, compared to a reference value for the expression level of the at least one molecule of interest.

In a particular embodiment, the subject not contacted with the non-integrative viral vector is the subject assessed for his/her ability to produce the at least one molecule. According to this embodiment, the reference value is the expression level of the at least one molecule in the serum of the subject not being incubated with the non-integrating viral vector.

The at least one molecule of interest may be endogenous to the subject, but the subject may suffer from a condition or disease caused by, or associated with, a decreased expression of the at least one molecule of interest.

In a particular embodiment, the reference value is the expression level of the at least one molecule in the serum of a healthy subject, i.e., of a subject with a normal level of expression of the at least one molecule.

Thus, in some embodiments, an expression level of the at least one molecule of interest increased up to 10%, 20%, 30%, 40%, 50%, 60%, 70%, 80%, 90%, or 100% of the normal or nominal expression level of the at least one molecule of interest in a healthy subject, means that the subject is able to produce the at least one molecule of interest. In some embodiments, an activity of the at least one molecule of interest increased up to 10%, 20%, 30%, 40%, 50%, 60%, 70%, 80%, 90%, or 100% of the normal or nominal activity of the at least one molecule of interest in a healthy subject, means that the subject is able to produce the at least one molecule of interest. Thus, in some embodiments, an expression level of the at least one molecule of interest increased from a range of value corresponding to a decreased expression up to the normal or nominal expression level of the at least one molecule of interest in a healthy subject, means that the subject is able to produce the at least one molecule of interest.

In some embodiments, the expression level and/or activity level of the at least one molecule of interest is substantially detectable by means known in the art.

In one embodiment, the non-integrating virus of the invention is an inactivated virus. By “inactivated virus”, it is meant that the virus lacks the ability to replicate. Thus, in one embodiment, the non-integrative viral vector is non-replicative or replication incompetent.

In some embodiments, the non-integrative viral vector is not a live virus. In some embodiments, the non-integrative viral vector is not a replication competent virus.

In some embodiments, the non-integrative viral vector has no, limited or acceptable side effects upon administration in a subject, preferably no side effects. In some embodiments, the non-integrative viral vector does not induce a disease in an individual.

In some embodiments, the non-integrative viral vector comprises at least one nucleic acid molecule encoding the at least one molecule of interest.

In one embodiment, the non-integrating virus vector of the invention is selected from the group comprising or consisting of adenovirus vector, adeno-associated virus (AAV) vector, integration-deficient lentiviral vector (IDLV), poxviral vector. In one embodiment, the non-integrating virus of the invention is not a lentivirus. In another embodiment, the non-integrating virus of the invention is not a retrovirus, such as a gammaretrovirus.

In some embodiments, the non-integrating virus vector is used in gene therapy or gene transfer.

As used herein, “gene therapy” refers to a therapeutic strategy aiming at expressing exogenously at least one gene in one or more cell, tissue or organ of a patient. The patient may be suffering from a condition or disease caused by at least one mutation on at least one gene, wherein the expression of the at least one gene corrects or alleviates the defects induced by the disease or condition. Typically, in a disease caused by a mutation resulting in the translation of a non-functional protein, gene therapy is used to express the gene encoding the functional protein. Non limitative examples of diseases caused by at least one mutation on at least one gene include genetic diseases and cancer. Alternatively, gene transfer may be used to exogenously express at least one protein from a pathogenic species (i.e., an antigen) enabling an immune response, preferably a lasting immune response, against this antigen; thus, gene transfer can be used for vaccination purposes. The term “gene therapy” is herein used interchangeably with the term “gene transfer”.

In some embodiments, the non-integrating virus vector is a DNA virus vector.

As used herein, DNA virus refers to an inactivated virus whose viral genome is made of double stranded DNA or single stranded DNA. In one embodiment, the DNA virus belongs to the family selected from the group comprising or consisting of Adenoviridae, Papovaviridae, Herpesviridae, Poxviridae, Pleolipoviridae, Parvoviridae, and Anelloviridae.

In some embodiments, the non-integrating virus belongs to the family selected from the group comprising or consisting of the Adenoviridae family and the Parvoviridae family.

In one embodiment, the non-integrating virus vector belongs to the Adenoviridae family.

In some embodiments, the non-integrating virus vector is an adenovirus vector.

Within the scope of this invention, adenovirus hereby refers to both natural and genetically modified viruses of the Adenoviridae family. In one embodiment, the adenovirus vector of the invention is a recombinant adenovirus vector.

In one embodiment, the adenovirus vector derives from a human or a non-human adenovirus. In some embodiments, the adenovirus vector is obtained from any one of human or non-human Ad serotypes.

In one embodiment, the adenovirus vector is obtained from any one of human Ad serotypes.

Human adenoviruses (HAd) comprise more than 60 identified serotypes, distributed among 7 species, namely HAd-A to HAd-G. Non limitative examples of human adenovirus serotypes commonly used for vector design include Ad serotypes 3, 4, 5, 6, 11, 26, 28 and 35 (HAd3, Had4, HAd 5, HAd 6, HAd 11, HAd 26, Had 28 and HAd 35). In some embodiments, the adenovirus vector is obtained from any one of human Ad serotypes. In some embodiments, the adenovirus vector is obtained from the Ad serotype selected from the group comprising or consisting of HAd3, Had4, HAd 5, HAd 6, HAd 11, HAd 26, Had 28 and HAd 35. In some preferred embodiments, the adenovirus vector is obtained from the Ad serotype selected from the group comprising or consisting of HAd 5, HAd6 and HAd 26.

In another embodiment, the adenovirus vector is obtained from any one of non-human Ad serotypes.

Non-human adenoviruses are also used for adenovirus vector design. Non limitative examples of non-human adenovirus serotypes used for vector design include bovine Ad serotype 3 (BAd3), canine Ad serotype 2 (CAd2), chimpanzee Ad serotypes 1, 2, 3, 5, 6, 7, 25 and 68 (ChAd1, ChAd2, ChAd3, ChAd5, ChAd6, ChAd7, ChAd25, ChAd68), ovine Ad serotype 7 (OAd7), porcine Ad serotype 3 and 5 (PAd3, PAd5) and fowl Ad serotypes 1, 4, 8, 9, and 10 (FAd1, Fad4, FAd8, FAd9, FAd10). In some embodiments, the adenovirus vector is obtained from the Ad serotype selected from the group comprising or consisting of BAd3, CAd2, ChAd1, ChAd2, ChAd3, ChAd5, ChAd6, ChAd7, ChAd25, ChAd68, OAd7, PAd3, PAd5, FAd1, Fad4, FAd8, FAd9 and FAd10. In some embodiments, the adenovirus vector is obtained from the Ad serotype selected from the group comprising or consisting of ChAd1, ChAd2, ChAd3, ChAd5, ChAd6, ChAd7, ChAd25 and ChAd68. In a preferred embodiment, the adenovirus vector is obtained from the Ad serotype ChAd25.

In another embodiment, the non-integrating virus vector belongs to the Parvoviridae family.

In some embodiments, the non-integrating virus vector is an adeno-associated virus (AAV) vector.

Within the scope of this invention, AAV hereby refers to both natural and genetically modified AAV. In one embodiment, the AAV vector of the invention is a recombinant AAV vector.

In one embodiment, the AAV vector derives from a human or a non-human AAV. In some embodiments, the AAV vector is obtained from any one of human or non-human AAV serotypes.

In some embodiments, the AAV vector is obtained from any of the following natural AAV serotypes, or derivatives thereof: AAV1, AAV3, AAV4, AAV5, AAV6, AAV7, AAV8, AAV9, AAV10, AAV11. In one embodiment, the AAV vector is obtained from the AAV serotype AAV3, or derivatives thereof.

In some embodiments, the AAV vector is not obtained from the AAV serotype AAV2, nor derivatives thereof.

As described above, the attachment receptor is specific of said non-integrating virus vector of the invention.

In some embodiments, the attachment receptor is selected from the group comprising or consisting of adenovirus attachment receptors and AAV attachment receptors.

In one embodiment, the attachment receptor is an adenovirus attachment receptor. In some embodiments, the adenovirus attachment receptor is selected from the group comprising or consisting of the coxsackievirus and adenovirus receptor (CAR), CD46, CD80, CD86, sialic acid and integrins. In a preferred embodiment, said adenovirus attachment receptor is CAR.

In certain embodiments, the adenovirus attachment receptor is transiently expressed in cultured cells.

Methods to transiently express a protein in cultured cells are known to the skilled in the art, and comprise but are not limited to electroporation, lipofection, nanoparticles, magnetic nanoparticles, sonoporation or injection of at least one nucleic acid molecule.

In certain embodiments, the adenovirus attachment receptor is naturally expressed in cultured cells. In certain embodiments, cultured cells are genetically modified to stably express the adenovirus attachment receptor.

Methods to stably express a protein in cultured cells are known to the skilled in the art, and comprise but are not limited to lentiviral vector infection, CIRSPR-Cas9 and transposon mutagenesis.

In some embodiments, the cultured cells are the CAR-expressing cells of a cell line selected from the group comprising A549, H460, Caco-2, HEK-293, NTERA-2, RT4, Hep G2, SuSa, AF22, HeCaT, BEWO, U-2 OS, HeLa, T-47d, hTCEpi, HAP-1, EFO-21, preferably A549.

In some embodiments, the cultured cells are the CAR-expressing cells of a cell line selected from the group comprising NTERA-2, RT4, Hep G2, SuSa, AF22, HeCaT, BEWO, U-2 OS, HeLa, T-47d, hTCEpi, HAP-1, EFO-21.

In a preferred embodiment, the cultured cells are the CAR-expressing cells of a cell line selected from the group comprising A549, H460, Caco-2, HEK-293.

In a preferred embodiment, the cultured cells are the CAR-expressing cells of cell line A549. In some embodiments, the cultured cells are the CAR-expressing cells of cell line H460. In some embodiments, the cultured cells are the CAR-expressing cells of cell line Caco-2. In some embodiments, the cultured cells are the CAR-expressing cells of cell line HEK-293.

In some embodiments, the cultured cells are transfected with at least one CAR-encoding nucleic acid molecule to transiently express CAR. In some embodiments, the cultured cells are genetically modified to express or overexpress CAR.

In some embodiments, the cells are primary cells. In a preferred embodiment, the cells are cells of an immortalized cell line.

In another embodiment, the attachment receptor is an AAV attachment receptor. In one embodiment, the AAV attachment receptor is a multiserotype AAV receptor (AAVR). In one embodiment, the AAV attachment receptor is a glycan receptor, such as heparan sulfate proteoglycan (HSPG), N-linked sialic acid moieties and N-linked galactose. In one embodiment, the AAV attachment receptor is a proteinaceous receptor, such as c-MET, FGFR1, A5β1 integrin, aVβ5 integrin, CD9 tetraspanin, EGFR, PDGFR and yeast two-hybrid screens (LamR).

In certain embodiments, the AAV attachment receptor is transiently expressed in cultured cells. In certain embodiments, the AAV attachment receptor is naturally expressed in cultured cells. In certain embodiments, cultured cells are genetically modified to stably express the AAV attachment receptor. In some embodiments, the cells are primary cells. In a preferred embodiment, the cells are cells of an immortalized cell line.

In some embodiments, the composition comprising the non-integrating virus vector is a vaccine composition or a pharmaceutical composition. In some embodiments, the non-integrating virus vector is comprised in a vaccine composition or in a pharmaceutical composition.

In one embodiment, the invention aims at assessing or detecting the resistance of a subject to a vaccine composition or a pharmaceutical composition comprising a non-integrating virus vector. In this embodiment, the present invention relates to an in vitro method for assessing subject resistance towards a vaccine composition or a pharmaceutical composition comprising a non-integrating virus vector, comprising the steps of (i) culturing cells expressing an attachment receptor specific of said non-integrating virus vector with a composition comprising said non-integrating virus vector and with a serum sample of said subject in a culture medium, and (ii) measuring the expression level of at least one molecule produced by said cultured cells.

In one embodiment, the composition comprising the non-integrating virus vector is a vaccine composition. As used herein, vaccine refers to any preparation comprising a substance or a group of substances meant to cause the immune system of an organism to respond to pathogens, such as bacteria or viruses, or to a tumor. Prophylactic vaccines are used to prevent a subject from ever having a particular disease or to only have a mild case of the disease. Such prophylactic vaccines usually comprise the pathogen responsible for the disease, either live and weakened, inactivated, or killed, or components thereof, purified or recombinant. Therapeutic vaccines are intended to treat specific diseases in a subject, in particular cancer. Such therapeutic anti-cancer vaccines comprise a tumor-antigen or tumor-antigens, eliciting an immune response directed against the tumor cells.

Non-limitative examples of the infectious disease include Anaplasmosis; Anthrax; Babesiosis; Botulism; Brucellosis; Burkholderia mallei infection (glanders); Burkholderia pseudomallei infection (melioidosis); Campylobacteriosis; Carbapenemresistant Enterobacteriaceae infection (CRE); Chancroid; Chikungunya infection; Chlamydia infection; Ciguatera; Clostridium difficile infection; Clostridium perfringens infection (Epsilon Toxin); Coccidioidomycosis fungal infection (Valley fever); Creutzfeldt-Jacob Disease, transmissible spongiform (CJD); Cryptosporidiosis; Cyclosporiasis; Dengue Fever; Diphtheria; E. Coli infection; Eastern Equine Encephalitis (EEE); Ebola Hemorrhagic Fever (Ebola); Ehrlichiosis; Arboviral or parainfectious encephalitis; Non-polio enterovirus infection; D68 enterovirus infection, (EV-D68); Giardiasis; Gonococcal infection (Gonorrhea); Granuloma inguinale; Type B Haemophilus influenza disease, (Hib or H-flu); Hantavirus pulmonary syndrome (HPS); Hemolytic uremic syndrome (HUS); Hepatitis A (Hep A); Hepatitis B (Hep B); Hepatitis C (Hep C); Hepatitis D (Hep D); Hepatitis E (Hep E); Herpes; Herpes zoster, zoster VZV (Shingles); Histoplasmosis; Human Immunodeficiency Virus/AIDS (HIV/AIDS); Human Papillomavirus (HPV); Influenza (Flu); Lead poisoning; Legionellosis (Legionnaires Disease); Leprosy (Hansens Disease); Leptospirosis; Listeriosis; Lyme Disease; Lymphogranuloma venereum infection (LVG); Malaria; Measles; Viral meningitis; Meningococcal disease; Middle East respiratory syndrome coronavirus 20 (MERS-CoV); Mumps; Norovirus; Paralytic shellfish poisoning; Pediculosis (lice, head and body lice); Pelvic inflammatory disease (PID); Pertussis; Bubonic, septicemic or pneumonic plague; Pneumococcal disease; Poliomyelitis (Polio); Psittacosis; Pthiriasis (crabs; pubic lice infestation); Pustular rash diseases (small pox, monkeypox, cowpox); Q-Fever; Rabies; Ricin poisoning; Rickettsiosis (Rocky Mountain Spotted Fever); Rubella, including congenital rubella (German Measles); Salmonellosis gastroenteritis infection; Scabies infestation; Scombroid; Severe acute respiratory syndrome (SARS); Shigellosis gastroenteritis infection; Smallpox; Methicillin-resistant Staphylococcal infection (MRSA); Staphylococcal food poisoning; Vancomycin intermediate Staphylococcal infection (VISA); Vancomycin resistant Staphylococcal infection (VRSA); Streptococcal disease, Group A; Streptococcal disease, Group B; Streptococcal toxic-shock syndrome (STSS); Primary, secondary, early latent, late latent or congenital syphilis; Tetanus infection (Lock Jaw); Trichinosis; Tuberculosis (TB); Latent tuberculosis (LTBI); Tularemia (rabbit fever); Typhoid fever, Group D; Typhus; Vaginosis; Varicella (chickenpox); Vibrio cholerae infection (Cholera); Vibriosis (Vibrio); Viral hemorrhagic fever (Ebola, Lassa, Marburg); West Nile virus infection; Yellow Fever; Yersinia infection and Zika virus infection.

In some embodiments, the vaccine is for immunization against a pathogen selected from the group comprising or consisting of Coronaviridae, Filoviridae, Orthomyxoviridae, Togaviridae, Pneumoviridae, Caliciviridae, Retroviridae, Flaviviridae, Mycobacteriaceae, and Plasmodium.

In one embodiment, the vaccine is for immunization against a virus of the family selected from the group comprising or consisting of Coronaviridae, Filoviridae, Orthomyxoviridae, Togaviridae, Pneumoviridae, Caliciviridae, or Retroviridae, Flaviviridae.

In some embodiments, the vaccine is for immunization against a virus of the family selected from the group comprising or consisting of Coronaviridae, Filoviridae, and Orthomyxoviridae.

In a preferred embodiment, the vaccine is for immunization against a virus of the Coronaviridae family. Non limitative examples of Coronaviridae viruses include Middle East respiratory syndrome-related coronavirus, human coronavirus 229E, human coronavirus NL63, human coronavirus OC43, human coronavirus HKU1, severe acute respiratory syndrome coronavirus 1 (SARS-CoV-1) and severe acute respiratory syndrome coronavirus 2 (SARS-CoV-2).

In one embodiment, the vaccine is for immunization against a virus of the Filoviridae family. Non limitative examples of Filoviridae viruses include Ebola virus, Marburg virus, Ravn virus, Tai Forest virus, Bundibugyo virus, Reston virus, Sudan virus and Bombali virus.

In one embodiment, the vaccine is for immunization against a virus of the Orthomyxoviridae family. Non limitative examples of Orthomyxoviridae viruses include Influenza A virus, Influenza B virus, Influenza C virus, Dhori virus, Bourbon virus and Quaranfil virus.

In one embodiment, the vaccine is for immunization against a virus of the Togaviridae family. Non limitative examples of Togaviridae viruses include Sindbis virus, Semliki Forest virus, Rio Negro virus, Mayaro virus Chikungunya virus, Eastern equine encephalitis virus, Ross River virus, O'nyong'nyong virus, and Barmah Forest virus.

In one embodiment, the vaccine is for immunization against a virus of the Pneumoviridae family. Non limitative examples of Pneumoviridae viruses include human respiratory syncytial virus (RSV) A2, human RSV B1, and human metapneumovirus.

In one embodiment, the vaccine is for immunization against a virus of the Caliciviridae family. Non limitative examples of Caliciviridae viruses include Norovirus and Sapovirus.

In one embodiment, the vaccine is for immunization against a virus of the Retroviridae family. Non limitative examples of Retroviridae viruses include human T-lymphotropic virus, human foamy virus, human immunodeficiency virus (HIV) type 1 and HIV type 2.

In one embodiment, the vaccine is for immunization against a virus of the Flaviviridae family. Non limitative examples of Flaviviridae viruses include hepatitis C virus (HCV), Zika virus, Dengue virus, West Nile virus (WNV), yellow fever virus.

In some embodiments, the vaccine is for immunization against a pathogen selected from the group comprising or consisting of Mycobacteriaceae, preferably Mycobacterium tuberculosis, and Plasmodium.

In one embodiment, the vaccine is for immunization against a bacterium of the Mycobacteriaceae family. In a preferred embodiment, said bacteria is Mycobacterium tuberculosis. Other, non-limitative examples of Mycobacterium pathogens include Mycobacterium leprae and Mycobacterium africanum.

In one embodiment, the vaccine is for immunization against a Plasmodium species selected from the group comprising Plasmodium falciparum: Plasmodium vivax, Plasmodium malariae, Plasmodium ovale, and Plasmodium knowlesi.

In one embodiment, the vaccine is Hexon/pVII-modified AdPfCSP.

In some embodiments, the vaccine is for immunization against a virus selected from the group comprising or consisting of SARS-CoV-2, Ebola virus, Chikungunya, human immunodeficiency virus (HIV), hepatitis C virus (HCV), Zika virus, respiratory syncytial virus (RSV), Norovirus, Influenza virus, preferably SARS-CoV-2.

In one embodiment, the vaccine is for immunization against a virus selected from the group comprising or consisting of SARS-CoV-2, Ebola virus or Influenza virus. In one embodiment, the vaccine is for immunization against Ebola virus.

In one embodiment, said vaccine is selected from the group comprising Ad5-EBOV, ChAd3-EBO-Z and Ad26.ZEBOV. In one embodiment, the vaccine is Ad5-EBOV. In one embodiment, the vaccine is ChAd3-EBO-Z. In one embodiment, the vaccine is Ad26.ZEBOV. In one embodiment, the vaccine is for immunization against Chikungunya. In one embodiment, the vaccine is for immunization against HIV.

In one embodiment, the vaccine is selected from the group comprising MRKAd5, MRKAd6, DNA/rAd5. In one embodiment, the vaccine is MRKAd5. In one embodiment, the vaccine is MRKAd6. In one embodiment, the vaccine is DNA/rAd5.

In one embodiment, the vaccine is for immunization against HCV. In one embodiment, the vaccine is for immunization against RSV. In one embodiment, the vaccine is for immunization against Zika virus. In one embodiment, the vaccine is GAd-Zvp. In one embodiment, the vaccine is for immunization against Norovirus. In one embodiment, the vaccine is for immunization against Influenza virus.

In one embodiment, the vaccine is selected from the group comprising cAdVax-FluAv, rAd-HA/H5N1/HongKong/156/97, AdCMV-PR8.ha, AdNCHA1.1, rAdH5/M2e.

In a preferred embodiment, the vaccine is for immunization against SARS-Cov-2.

In certain embodiments, the vaccine is selected from the group comprising ChAdOx1 nCoV-19 (AstraZeneca), Ad26.COV2.S (Johnson & Johnson), Gam-COVID-Vac/Sputnik V (Gamaleya Research Institute of Epidemiology and Microbiology), ImmunityBio COVID-19/hAd5 (ImmunityBio), AD5-nCOV/Convidecia (CanSino Biologics).

In certain embodiments, the vaccine is selected from the group comprising ChAdOx1 nCoV-19, Ad26.COV2.S, Gam-COVID-Vac/Sputnik V. In one embodiment, the vaccine is Ad26.COV2.S. In one embodiment, the vaccine is Gam-COVID-Vac/Sputnik V. In a preferred embodiment, the vaccine is ChAdOx1 nCoV-19.

Thus, in some embodiments, the non-integrating virus vector comprises at least one nucleic acid molecule encoding at least one antigen from a pathogen as described hereinabove.

In another embodiment, the composition comprising the non-integrating virus vector is a pharmaceutical composition. In some embodiments, the pharmaceutical composition comprising the non-integrating virus vector further comprises an adjuvant or a pharmaceutically active agent.

In some embodiments, the pharmaceutical composition is for treating or preventing a disease.

In some embodiments, the pharmaceutical composition is for treating or preventing a disease selected from the group comprising or consisting of cancer and genetic diseases.

In some embodiments, the pharmaceutical composition is for treating or preventing cancer.

Within the scope of the invention, the term “cancer” is intended to refer to, or to describe, the physiological condition in mammals that is typically characterized by unregulated cell growth or proliferation. Examples of cancer include, but are not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, colorectal cancer, endometrial carcinoma, salivary gland carcinoma, kidney cancer, vulvar cancer, thyroid cancer, hepatic carcinoma and various types of head and neck cancer.

In some embodiments, the pharmaceutical composition comprising the non-integrating virus vector further comprises an anticancer agent.

Non limitative examples of adjuvants include aluminum, AS01, AS03, AS04, MF59, AS01B, CpG 1018, Matrix-M, TLR7/TLR8 ligand, ISA51.

Non-limitative examples of pharmaceutically active anticancer agents include acalabrutinib, alectinib, alemtuzumab, anastrozole, avapritinib, avelumab, belinostat, bevacizumab, bleomycin, blinatumomab, bosutinib, brigatinib, carboplatin, carmustine, cetuximab, chlorambucil, cisplatin copanlisib, cytarabine, daunorubicin, decitabine, dexamethasone, docetaxel, doxorubicin, encorafenib, erdafitinib, etoposide, everolimus, exemestane, fludarabine, 5-fluorouracil, gemcitabine, ifosfamide, imatinib Mesylate, leuprolide, lomustine, mechlorethamine, melphalan, methotrexate, mitomycin, nelarabine, paclitaxel, pamidronate, panobinostat, pralatrexate, prednisolone, ofatumumab, rituximab, temozolomide, topotecan, tositumomab, trastuzumab, vandetanib, vincristine, vorinostat, zanubrutinib, and the likes.

In some embodiments, the non-integrating virus vector comprises at least one nucleic acid molecule encoding at least one antitumor molecule and/or at least one molecule inhibiting oncogenes. Non-limitative examples include tumor suppressors such as p53, or interfering RNA against an oncogene.

In some embodiments, the pharmaceutical composition is for treating or preventing a genetic disease.

In some embodiments, the genetic disease is selected from the group comprising or consisting of hemophilia, cystic fibrosis, albinism, Duchenne muscular dystrophy, Huntington disease, Parkinson's disease, Spinal Muscular Atrophy (SMA), Fabry disease, Gaucher disease, Batten Disease, Choroideremia, Retinitis pigmentosa, sickle cell disease, thalassemia, fragile X syndrome, Charcot-Marie-Tooth disease, mucolipidosis, Wilson disease, severe combined immune deficiency and Hunter syndrome.

In some embodiments, the non-integrating virus vector comprises at least one nucleic acid molecule encoding at least one protein defective in a genetic disease.

In some embodiments, the genetic disease is hemophilia.

In some embodiments, the non-integrating virus vector used for the treatment of the genetic disease comprises at least one functional copy of a gene that is mutated and/or non-functional in the patient suffering from the genetic disease. In some embodiments, the expression of the at least one gene comprised in the non-integrating virus vector corrects the defect causing the genetic disease.

Non limitative examples of gene for treating genetic diseases include the gene of factor VIII in hemophilia A, factor IX in hemophilia B or factor XI in hemophilia C.

In some embodiments, the serum sample is obtained from a blood sample of a subject after centrifugation. In certain embodiments, the serum sample is obtained from a plasma sample of a subject after centrifugation.

In some embodiments, the cells cultured in step (i) are adherent and are seeded on a suitable support, e.g., the wells of a culture plate or a culture flask. In certain embodiments, the cultured cells are non-adherent and are seeded in a suitable vessel, e.g., a suspension culture flask or tube. In a preferred embodiment, the cultured cells are adherent and are seeded on a support selected from the group comprising 4-well cell culture plates, 6-well cell culture plates, 8-well cell culture plates, 12-well cell culture plates, 24-well cell culture plates, 48-well cell culture plates, 96-well cell culture plates, 384-well cell culture plates and 1536-well cell culture plates. In a more preferred embodiment, the cultured cells are seeded on a 24-well cell culture plate.

In some embodiments, the cultured cells are counted prior to seeding, and diluted in order to attain a specific seeding density. In some embodiments, a seeding concentration is calculated according to both the specific seeding density and the support selected for seeding. In certain embodiments, the seeding concentration is comprised between about 10,000 and about 10,000,000 cells/mL. In a preferred embodiment, the seeding concentration is comprised between about 50,000 and about 500,000 cells/mL. In a more preferred embodiment, the seeding concentration is about 125,000 cells/mL.

In some embodiments, the non-integrating virus vector and/or the serum sample are used pure, i.e., non-diluted. In another embodiment, the non-integrating virus vector and/or the serum sample are diluted in an appropriate aqueous solvent.

In certain embodiments, the non-integrating virus vector is diluted in an aqueous solvent. In a preferred embodiment, the non-integrating virus vector is diluted in a culture medium. In a more preferred embodiment, the non-integrating virus vector is diluted in the same culture medium as the cultured cells.

In some embodiments, the non-integrating virus vector is diluted by a dilution factor comprised between 10-fold and 10,000-fold. As used herein, the expression “between 10-fold and 10,000-fold” encompasses 10-fold, 11-fold, 12-fold, 13-fold, 14-fold, 15-fold etc., until 10,000-fold.

In some embodiments, the non-integrating virus vector is diluted by a dilution factor comprised between 100-fold and 5,000-fold. In a preferred embodiment, the non-integrating virus vector is diluted by a dilution factor of 1,000-fold.

In certain embodiments, the serum sample is diluted in an aqueous solvent. In a preferred embodiment, the serum sample is diluted in a culture medium. In a more preferred embodiment, the serum sample is diluted in the same culture medium as the cultured cells.

In one embodiment, the culture medium is Dulbecco's Modified Eagle Medium (DMEM).

In some embodiments, the serum sample is diluted by a dilution factor comprised between 2-fold and 10,000-fold.

As used herein, the expression “between 2-fold and 10,000-fold” encompasses 2-fold, 3-fold, 4-fold, 5-fold etc., until 10,000-fold.

In some embodiments, the serum sample is diluted by a dilution factor comprised between 10-fold and 5,000-fold. In some embodiments, the serum sample is diluted by a dilution factor comprised between 4-fold and 1,024-fold.

In some embodiments, several dilutions of the serum sample are performed. In some embodiments, serial dilutions of the serum sample are prepared, with a dilution step comprised between 2-fold and 10-fold between each serial dilution. In a preferred embodiment, the dilution step between each serial dilution is 2-fold.

In some embodiments, the composition comprising the non-integrating virus vector and the serum sample are brought into contact with the cells after seeding of the cells. In another embodiment, the composition comprising the non-integrating virus vector and the serum sample are mixed with the cells prior seeding the cells.

In some embodiments, the composition comprising the non-integrating virus vector and the serum sample are added separately to the cells. In one embodiment, the serum sample is added to the cells before the composition comprising the non-integrating virus vector. In another embodiment, the composition comprising the non-integrating virus vector is added to the cells before the serum sample.

In some embodiments, step (ii) is performed during a period of about 3 to about 15 days.

Within the scope of the invention, the expression “about 3 to about 15 days” encompasses 3, 4, 5, 6, 7, 8, 9, 10, 11, 12, 13, 14 and 15 days.

In a preferred embodiment, step (ii) is performed during at least 5 days. In a more preferred embodiment, step (ii) is performed during about 7 days.

In some embodiments, step (ii) is performed during at most about 7 days.

It will be apparent to the person skilled in the art how to adapt the incubation period depending on the cell type and the non-integrative viral vector type used, by performing routine checks for measuring, e.g., viability.

In some embodiments, the duration of step (ii) is adjusted within the scope of the invention according to the results of a pre-test which consists in assessing cell death.

Cell death can be assessed by persons skilled in the art, using techniques including, but not limited to, vital dyes staining, e.g., propidium iodide or calcein-AM, enzymatic activity detection, e.g., caspase, glucose-6-phosphate dehydrogenase or lactate dehydrogenase, assessment of redox state, e.g., by measuring resazurin conversion, assessment of metabolic activity, e.g., by measuring uptake of tetrazolium salts, cell detachment, immunodetection of a cell death marker, e.g., extracellular phosphatidylserine.

In some embodiments, the cell culture of step (i) and/or step (ii) is performed between 30° C. and 40° C., preferably at 37° C., in presence of between 1% and 10% of carbon dioxide, preferably in presence of 5% of carbon dioxide.

In some embodiments, the cell culture of step (i) and/or step (ii) is performed at an oxygen tension of about 21%.

In one embodiment, the culture medium is any culture medium designed to support the growth of the cells known to one of ordinary skill in the art. As used herein, such culture medium is called “proliferation medium” or “growth medium”. Examples of growth medium include, without limitation, MEM, DMEM, IMDM, RPMI 1640, FGM or FGM-2, 199/109 medium, HamF10/HamF12 or McCoy's 5A. In a preferred embodiment, the culture medium is DMEM or RPMI.

In one embodiment, the culture medium may further comprise any supplementary factors known by the person skilled in the art that may be used in cell culture. Examples of supplementary factors include, but are not limited to, FBS; glycine; amino acids, such as glutamine, asparagine, glutamic acid, aspartic acid, serine, proline or alanine, preferably the L-configuration of amino acids; and antibiotics, such as streptomycin or penicillin.

In some embodiments, the at least one molecule of interest is intracellular, periplasmic or extracellular. In a preferred embodiment, the at least one molecule of interest is periplasmic or extracellular. In a more preferred embodiment, the at least one molecule of interest is extracellular.

In some embodiment, the at least one molecule of interest is detected in the supernatant. In some embodiments, the at least one molecule of interest is free in the supernatant and/or comprised in an extracellular vesicle or exosome.

In some embodiment, the at least one molecule of interest is not intracellular.

In some embodiment, the at least one molecule of interest is not a fluorescent molecule.

In some embodiments, the at least one molecule produced by said cultured cells is a protein, polypeptide or peptide encoded by at least one nucleic acid within the non-integrating virus vector.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified by, for example, disulfide bond formation, glycosylation, lipidation, acetylation, phosphorylation, pegylation, or any other manipulation, such as conjugation with a labelling component. As used herein the term “amino acid” includes natural and/or unnatural or synthetic amino acids, including glycine and both the D or L optical isomers, and amino acid analogs and peptidomimetics.

In certain embodiments, the at least one molecule produced by said cultured cells is a peptide or a polypeptide encoded by at least one nucleic acid within the non-integrating virus vector.

In a preferred embodiment, the at least one molecule produced by said cultured cells is a protein encoded by at least one nucleic acid within the non-integrating virus vector.

In some embodiments, the expression level of the at least one molecule is measured at the protein level.

In certain embodiments, the measurement is performed by the detection and/or quantification of the at least one protein. In one embodiment, the at least one protein is detected and/or quantified in the cultured cells preparation.

In some embodiments, the method used to detect and/or quantify the at least one protein is selected from the group comprising enzyme-linked immunosorbent assay (ELISA), Western blot, Dot blot, immunofluorescence, immunochemistry, immunoprecipitation, fluorescent activated cell sorting (FACS), detection of at least one tag on the at least one protein, high-performance liquid chromatography (HPLC), and liquid chromatography-mass spectrometry (LC-MS).

In a preferred embodiment, the method used to detect and/or quantify the at least one protein is ELISA.

In some embodiments, the at least one protein is detected in a cell lysate of the cultured cell preparation. In certain embodiments, the cell lysate is diluted in a solution suitable for the detection technique. In some embodiments, the lysate is further added with at least one agent for preventing protein degradation, e.g., protease inhibitors.

Cell lysis methods are known to the skilled in the art and comprise, but are not limited to, mechanical disruption, e.g., using beads, osmotic shock, freeze/thaw, sonication, enzyme digestion, detergent e.g., SDS or Triton X-100.

In a preferred embodiment, the at least one protein is detected in the supernatant of the cultured cell preparation. In certain embodiments, the supernatant is diluted in a solution suitable for the detection technique. In certain embodiments, the supernatant is centrifuged. In some embodiments, the supernatant is further added with at least one agent for preventing protein degradation, e.g., protease inhibitors.

In some embodiments, the at least one molecule is not a fluorescent protein such as GFP, RFP, YFP and the like.

In some embodiments, the at least one molecule is a protein naturally found in pathogenic species selected from the group comprising viruses, bacteria, mycobacteria and eukaryote parasites.

In some embodiments, the at least one molecule is a mycobacterial protein. In certain embodiments, the at least one molecule is a bacterial protein. In certain embodiments, the at least one molecule is protein from a eukaryote parasite. In a preferred embodiment, the at least one molecule is a viral protein.

In certain embodiments, the viral protein is selected from the group comprising the viral protein Spike (S), the Ebola virus glycoprotein (EBOV GP), Hemagglutinin (HA), Glycoprotein 41 and 120 (Gp41, Gp120), the hepatitis C virus NS antigen, CHIKV full-length structural polyprotein (Capsid, E3, E2, 6k and E1), the respiratory syncytial virus (RSV)-fusion protein or the GI.1 and GII.4 antigen protein from norovirus.

In a preferred embodiment, the viral protein is selected from the group comprising the viral protein Spike (S), the Ebola virus glycoprotein (EBOV GP), Hemagglutinin (HA).

In a preferred embodiment, the viral protein is Spike (S). In one other embodiment, the viral protein is EBOV GP. In one other embodiment, the viral protein is HA. In one other embodiment, the viral protein is selected from the group comprising Gp41 and Gp120. In one other embodiment, the viral protein is the hepatitis C virus NS antigen. In one other embodiment, the viral protein is selected from the group comprising Capsid, E3, E2, 6k and E1. In one other embodiment, the viral protein is the RSV-fusion protein. In one other embodiment, the viral protein is selected from the group comprising the GI.1 and GII.4 antigen protein from norovirus. In one other embodiment, the at least one protein is Falciparum circumsporozoite surface antigen. In one other embodiment, the at least one protein is selected from the group comprising mycobacterial antigens 85A, 85B, and TB10.4.

In some embodiments, the at least one molecule is a nucleic acid molecule.

As used herein, nucleic acid molecule refers to either desoxyribonucleic acid (for example, but not limited to, a cDNA or genomic DNA) or ribonucleic acid (for example, but not limited to, a mRNA or tRNA). The nucleic acid molecule can be single-stranded or double-stranded.

In some embodiments, the nucleic acid molecule is comprised in the non-integrating virus vector sequence.

In some embodiments, the nucleic acid molecule is produced in the cells cultured in the presence of the non-integrating virus vector. In a preferred embodiment, the nucleic acid molecule is produced by the cells from the non-integrating virus vector sequence. In one embodiment, the nucleic acid molecule is replicated using the non-integrating virus vector sequence as a template. In another embodiment, the nucleic acid molecule is transcribed from the non-integrating virus vector sequence.

In one embodiment, the nucleic acid molecule is detectable in cells exposed to the non-integrating virus vectors. In another embodiment, the nucleic acid molecule is detectable in cells that have previously been, but no longer are, exposed to the non-integrating virus vector.

In some embodiments, the expression level of the at least one nucleic acid molecule is measured at the RNA level.

In a preferred embodiment, the method used to measure the at least one RNA molecule expression is selected from the group comprising RNA-seq, microarrays, RT-PCR, RT-qPCR, Northern Blot and/or hybridization techniques.

In a preferred embodiment, the method used to measure the at least one RNA molecule expression is RT-qPCR. In a preferred embodiment, the method used to measure the at least one RNA molecule expression is RNA-seq.

In some embodiments, the at least one RNA molecule is extracted from the cultured cells. In one embodiment, the cells are disrupted and the at least one RNA molecule is purified on an exclusion column. In certain embodiments, the RNA extract is treated with DNAse to avoid DNA contamination. In certain embodiments, RNA is treated with agents preventing RNA degradation, e.g., RNAse inhibitors.

RNA extraction and purification methods are known to the skilled in the art and are widely available in the form of commercial kits.

In one embodiment, the in vitro method of the present invention is also a method of identifying the subject as being resistant to the non-integrating virus vector, or determining if the subject is resistant to the non-integrating virus vector. In one embodiment, the subject is identified as being resistant to the non-integrating virus vector if the expression level of the at least one molecule is substantially superior to the reference value.

In some embodiments, the ability of the subject to produce the at least one therapeutic molecule of interest encoded by the non-integrative viral vector means that the subject is not resistant to the non-integrative viral vector.

In some embodiments, the inability of the subject to produce the at least one therapeutic molecule of interest encoded by the non-integrative viral vector means that the subject is resistant to the non-integrative viral vector.

Accordingly, another object of the invention is an in vitro method for determining if the subject is resistant to a non-integrating virus vector, comprising the steps of:
(i) culturing cells expressing an attachment receptor specific of the non-integrating viral vector;
(ii) incubating the cells with a composition comprising the non-integrating viral vector and a serum sample of the subject in a culture medium;
(iii) measuring the expression level of the at least one molecule of interest encoded by the non-integrative viral vector;
(iv) comparing the expression level measured in step (iii) with a reference value;
(v) determining if the subject is resistant to the non-integrating virus vector, wherein an expression level of the at least one molecule of interest superior to a reference value means that the subject is not resistant to the non-integrating virus vector.

In some embodiments, the subject is resistant when the at least one molecule is superior to a reference value, as measured by the appropriate detection technique according to the in vitro method used to measure the expression level of the at least one molecule produced by the cultured cells in step (iii).

As used herein, substantially undetectable means that the levels of the molecule are below the detection threshold of the in vitro method, wherein the threshold refers to a reference value to which the expression level measured in step (iii) is compared.

In some embodiments, a calibration step is performed before measuring the at least one molecule.

In some embodiments, the parameters of the chosen detection technique are adjusted in order to bring specific concentrations within the detection range, wherein specific concentrations correspond to the ones that are expected to be found in the cultured cells of the invention.

In some embodiments, the expression level measured in step (iii) of the invention is compared to the expression level measured with at least one reference sample, wherein the at least one reference sample is selected from the group comprising:
a serum sample from a subject known to be responsive to the vaccine composition or the pharmaceutical composition comprising the non-integrating virus vector,
a culture medium, preferably the same culture medium used to culture the cells during step (i) and/or step (ii),
a serum sample from a subject known to have an immune resistance to the non-integrating virus vector tested,
a composition comprising antibodies directed specifically against at least one antigen on the non-integrating virus vector.

In certain embodiments, the non-integrating virus vector is the same as the one that the subject has been treated with or will be treated with.

In one embodiment, the subject has already been submitted to a composition comprising an non-integrating virus vector. In this embodiment, the subject may be known to be resistant to the non-integrating virus vector. For example, in one embodiment, the subject is known to be resistant to the vaccine composition or the pharmaceutical composition comprising the non-integrating virus vector; in such case, the subject may be treated with immunosuppressive agents, e.g., glucocorticoids.

In another embodiment, the subject is susceptible to be submitted to or administered with a composition comprising an non-integrating virus vector. In this embodiment, it may be not known whether the subject is resistant or not to the non-integrating virus vector.

In one embodiment, the subject is a mammal, preferably a human. In one embodiment, the subject is a female. In another embodiment, the subject is a male.

In one embodiment, the subject is 18-years old or more. In another embodiment, the subject is under 18-years old.

The present invention further relates to an in vitro method for assessing the capacity or ability of a non-integrative viral vector encoding at least one molecule of interest to induce the production of the at least one molecule of interest in a subject, comprising the steps of:
(i) culturing cells expressing an attachment receptor specific of the non-integrating viral vector;
(ii) incubating the cells with a composition comprising the non-integrating viral vector and a serum sample of the subject in a culture medium; and
(iii) measuring the expression level of the at least one molecule of interest,
   wherein an expression level of the at least one molecule of interest superior to a reference value means that the non-integrative viral vector is capable or able of producing the at least one molecule of interest in the subject.

The present invention further relates to an in vitro method for screening non-integrative viral vectors that encode at least one molecule comprising the steps of:
(i) culturing cells expressing an attachment receptor specific of the non-integrating viral vector;
(ii) incubating the cells with a composition comprising the non-integrating viral vector and a serum sample of a subject in a culture medium; and
(iii) measuring the expression level of the at least one molecule of interest.

In some embodiments, the screening comprises the screening of more than one type of non-integrative viral vectors, typically a serum sample of a subject is tested for non-integrative viral vectors of different species and/or serotypes in order to determine at least one non-integrative viral vector for which the subject is not resistant.

In some embodiments, the screening is performed prior to the administration of the non-integrative viral vector to the subject.

The present invention further relates to an in vitro method of assessing the efficacy of a non-integrative viral vector to produce at least one molecule in a subject comprising the steps of:
(i) culturing cells expressing an attachment receptor specific of the non-integrating viral vector;
(ii) incubating the cells with a composition comprising the non-integrating viral vector and a serum sample of the subject in a culture medium; and
(iii) measuring the expression level of the at least one molecule of interest,
   wherein an expression level of the at least one molecule of interest superior to a reference value means that the non-integrative viral vector is effective to produce the at least one molecule of interest in the subject.

The present invention further relates to an in vitro method for assessing a subject's resistance towards a non-integrating virus vector, comprising the steps of (i) culturing cells expressing an attachment receptor specific of the non-integrating virus vector with a composition comprising the non-integrating virus vector and with a serum sample of the subject in a culture medium, and (ii) measuring the expression level of at least one molecule produced by the cultured cells, wherein the expression of the at least one molecule superior to a reference value means that the subject is not resistant towards said non-integrating virus vector.

The present invention further concerns an in vitro method of diagnosing resistance to a non-integrating virus vector in a subject, the method comprising the steps of (i) culturing cells expressing an attachment receptor specific of said non-integrating virus vector with said composition comprising the non-integrating virus vector and with a serum sample of said subject in a culture medium, and (ii) measuring the expression level of at least one molecule produced by said cultured cells.

In one embodiment, the in vitro method of the present invention comprises a step of diagnosing the subject as being resistant to said non-integrating virus vector. In one embodiment, the subject is diagnosed as being resistant to said non-integrating virus vector if the expression level of the at least one molecule is substantially undetectable.

The present invention enables the prediction or identification of the risk that a treatment comprising administration of a composition comprising a non-integrating virus vector may not be successful in a subject. In other words, the invention enables the prediction or identification of the risk of inefficacy of a composition comprising a non-integrating virus vector in a subject.

Another object of the present invention is an in vitro method of predicting the risk of inefficacy of a treatment comprising administration of a composition comprising an non-integrating virus vector in a subject susceptible to be administered with this composition, the method comprising the steps of (i) culturing cells expressing an attachment receptor specific of said non-integrating virus vector with said composition comprising the non-integrating virus vector and with a serum sample of said subject in a culture medium, and (ii) measuring the expression level of at least one molecule produced by said cultured cells.

In one embodiment, the treatment is considered as being at risk of being ineffective in a subject, if the at least one molecule is substantially undetectable.

The invention also enables to distinguish if the resistance to a non-integrating virus vector is due to lymphocytic or myolitic defect, or if it is due to pre-existing immune resistance to non-integrating virus vectors.

The present invention thus also relates to an in vitro method of identifying the cause of the resistance of a subject known to be resistant to a composition comprising an non-integrating virus vector, comprising the steps of (i) culturing cells expressing an attachment receptor specific of said non-integrating virus vector with said composition comprising the non-integrating virus vector and with a serum sample of said subject in a culture medium, and (ii) measuring the expression level of at least one molecule produced by said cultured cells.

If the resistance to the non-integrating virus vector has a lymphocytic or myolitic origin, the at least one molecule should be produced. On the contrary, if the resistance to the non-integrating virus vector is due to a pre-existing immune resistance to adenovirus vectors, the at least one molecule should not be produced.

In one embodiment, the resistance of a subject is considered as having a lymphocytic or myolitic origin, if the at least one molecule is substantially undetectable.

In some embodiments, the resistance to non-integrating virus vectors is a pre-existing immune resistance to non-integrating virus vectors.

As used herein, immune resistance to non-integrating virus vectors relates to both innate immunity and adaptative immunity responses to at least one specific antigen on the non-integrating virus vector, wherein said response is directed specifically to the at least one antigen on said non-integrating virus vector. Preferably, the immune response induces the neutralization of the targeted antigen. More preferably, the immune response induces the degradation of the targeted antigen.

Exemplary immune responses include T cell responses, e.g., cytokine production and cellular cytotoxicity, in addition, the term immune response includes immune responses that are indirectly affected by T cell activation, e.g., antibody production (humoral responses) and activation of cytokine responsive cells, e.g., macrophages. Immune cells involved in the immune response include lymphocytes, such as B cells and T cells (CD4+, CD8+, Th1 and Th2 cells); antigen presenting cells (e.g., professional antigen presenting cells such as dendritic cells); natural killer cells; myeloid cells, such as macrophages, eosinophils, mast cells, basophils, and granulocytes. For instance, immune responses are involved in transplant rejection, as well as in the concomitant physiological result of such immune responses, such as for example, interstitial fibrosis, chronic graft arteriosclerosis, or vasculitis. Immune responses are also involved in autoimmune diseases and the concomitant physiological result of such immune responses, including T cell-dependent infiltration and direct tissue injury, T cell-dependent recruitment and activation of macrophages and other effector cells, and T cell-dependent B cell responses leading to autoantibody production.

In certain embodiments, the pre-existing immune resistance to non-integrating virus vectors is conferred by humoral adaptative immunity and/or cellular adaptative immunity.

By humoral adaptative immunity, it is meant at least one circulating antibody clone directed specifically against the at least one antigen on said non-integrating virus vector.

By cellular adaptative immunity, it is meant at least one lymphocyte clone directed specifically against the at least one antigen on said non-integrating virus vector, wherein said lymphocyte is selected from the group consisting of B cells, memory B cells, helper T cells, cytotoxic T cells and memory T cells.

The existence of an immune resistance to non-integrating virus vectors in a subject can be caused by the following: previous exposition to at least one non-integrating virus, or part of the at least one non-integrating virus, sharing at least one common antigen with the non-integrating virus vector; previous exposition to at least one other non-integrating virus vector, or part of said at least one other non-integrating virus vector, sharing at least one common antigen with the non-integrating virus vector; transfusion with blood from a human with immune resistance to non-integrating virus vectors; previous exposition to any virus other than an non-integrating virus, or vaccine other than non-integrating virus vector-based vaccine, sharing at least one common antigen with the non-integrating virus vector.

In some embodiments, the detection of a pre-existing immune resistance towards non-integrating virus vectors in a subject may enable the prediction of the efficacy of the treatment using the non-integrating virus vector. In some embodiments, the detection of a pre-existing immune resistance towards non-integrating virus vectors in a subject may indicate not to use a non-integrating virus vector-based treatment.

The present invention further relates to an in vitro method to assess the susceptibility of a subject to respond to a treatment with a non-integrative viral vector encoding at least one molecule of interest, comprising the steps of:
(i) culturing cells expressing an attachment receptor specific of the non-integrating viral vector;
(ii) incubating the cells with a composition comprising the non-integrating viral vector and a serum sample of the subject in a culture medium;
(iii) measuring the expression level of the at least one molecule of interest; and
(iv) assessing if the subject is susceptible to respond to the treatment, wherein an expression level of the at least one molecule of interest superior to a reference value means that the subject is susceptible to respond to the treatment.

In some embodiments, the treatment is selected from the group comprising or consisting of vaccination, anticancer or antitumor therapy, gene therapy, and hemophilia therapy.

The present invention further relates to an in vitro method for identifying if a subject is at risk of developing an unwanted or adverse effect upon administration of a non-integrative viral vector encoding at least one molecule of interest, comprising the steps of:
(i) culturing cells expressing an attachment receptor specific of the non-integrating viral vector;
(ii) incubating the cells with a composition comprising the non-integrating viral vector and a serum sample of the subject in a culture medium; and
(iii) measuring the expression level of the at least one molecule of interest
(iv) identifying if the subject is at risk of developing an unwanted or adverse effect, wherein an expression level of the at least one molecule of interest superior to a reference value means that the subject is at risk of developing an unwanted or adverse effect upon administration of a non-integrative viral vector.

In some embodiments, the unwanted or adverse effect is selected from the group comprising or consisting of dyspnea, thrombocytopenia, thrombosis, vaccine-induced immune thrombotic thrombocytopenia (VITT), ischemia, stroke, allergy, anaphylaxis, headache, fever, nausea, tiredness, and combination thereof. In some embodiments, the unwanted or adverse effect is selected from the group comprising or consisting of dyspnea, thrombocytopenia, thrombosis, and VITT.

In some embodiments, the unwanted or adverse effect is associated with at least one clinical parameter selected from the group comprising or consisting of low platelet count, increased D-dimers plasma levels, low plasma fibrinogen, presence of anti-PF4 antibodies, and combination thereof.

In one embodiment, low platelet count means less than 150,000 platelets per μL of blood. In one embodiment, increased D-dimers plasma levels means more than 5,000 ng/mL. In some embodiments, low plasma fibrinogen means less than 500 mg/dL.

The present invention further relates to a method for preventing and/or treating a disease with a non-integrative viral vector encoding at least one molecule of interest in a subject, comprising the steps of:
determining if the subject is susceptible to respond to a treatment with the non-integrative viral vector encoding the at least one molecule of interest by:
   (i) culturing cells expressing an attachment receptor specific of the non-integrating viral vector;
   (ii) incubating the cells with a composition comprising the non-integrating viral vector encoding the at least one molecule of interest, and a serum sample of the subject in a culture medium;
   (iii) measuring the expression level of the at least one molecule of interest; and
   (iv) determining if the subject is susceptible to respond to a treatment with the non-integrative viral vector, wherein an expression level of the at least one molecule of interest superior to a reference value means that the subject is susceptible to respond,
administering to the subject susceptible to respond a therapeutically effective amount of the non-integrative viral vector encoding at least one molecule of interest.

In some embodiments, the method further comprises co-administering to the subject at least one pharmaceutically acceptable excipient along with the non-integrative viral vector encoding at least one molecule of interest. In some embodiments, the pharmaceutically acceptable excipient is an adjuvant.

In some embodiments, the step of administering to the subject a therapeutically effective amount of the non-integrative viral vector encoding at least one molecule of interest is repeated at least once, twice, or more.

In one embodiment, the method is for preventing an infectious disease. In some embodiments, the method for preventing an infectious disease is a vaccination method.

In some embodiments, the at least one molecule of interest is an antigen from a pathogen species or from a tumor. In some embodiments, the at least one molecule of interest is immunogenic.

In another embodiment, the method is for treating a genetic disease.

In some embodiments, the at least one molecule of interest is a molecule defective in the genetic disease. As used herein, “defective” means its expression level and/or activity is 70%, 60%, 50%, 40%, 30%, 20%, 10%, 5%, 1% or 0% of the normal or nominal expression level and/or activity of the molecule in a healthy subject.

In another embodiment, the method is for treating and/or preventing a cancer.

In some embodiments, the at least one molecule of interest is a molecule downregulated in cancer, typically a tumor suppressor. In some embodiments, the at least one molecule of interest is an inhibitor of a molecule upregulated in cancer, typically an inhibitor of an oncogene. Suitable tumor suppressors and oncogene targets are well known to the person skilled in the art.

The present invention further relates to a kit for implementing the in vitro method. In one embodiment, the kit comprises means for the determination or measurement of said at least one molecule.

In some embodiments, the kit further comprises at least one antibody specific for the at least one molecule, wherein said at least one molecule is a protein.

In some embodiments, the kit further comprises at least one nucleic acid molecule complementary to the least one molecule, wherein said at least one molecule is a nucleic acid molecule.

In some embodiments the kit further comprises an aqueous solution suitable for the dilution of the sample to be tested, wherein said sample is a culture medium supernatant, a cell lysate or a nucleic acid extract, preferably a culture medium supernatant. In certain embodiments, said aqueous solution is selected from the group comprising a biological buffer solution, a culture medium, and ultra-pure water.

### BRIEF DESCRIPTION OF THE DRAWINGS

FIG. 1 is a histogram showing the absorbance at 450 nm of controls sera and subject serum on day 0 and day 1 of hospitalization.

FIG. 2 is a histogram showing the concentration of S protein of controls sera and subject serum on day 0 and day 1 of hospitalization.

### EXAMPLES

The present invention is further illustrated by the following examples.

### Example 1

### Materials and Methods

### Materials

The materials used in the method of the invention comprise:
A549 cells, expressing the CAR receptor and thus allowing interaction with the vaccine;
Active Motif ELISA kit for the detection of protein S allowing the determination of the protein in the supernatant (La Hulpe, Belgium);
VAXZEVRIA® vaccine, allowing the transfection of cells and the production of protein S;
Dulbecco's Modified Eagle Medium (DMEM) with L-glutamine. This medium is used as a culture medium;
Serum sample of COVID-19 subject;
Serum sample of VAXZEVRIA 2 doses respondent subjects (the response to the vaccine was quantified on the Roche Cobas 8000 with the anti-S).

### Method

Day 1: 400 μL of a dilution containing a 125 000 cells/mL, is dispensed per well in a 24-well plate. This cells concentration is the optimal concentration to achieve confluence in 24-well plates with minimum lethality (Sci Rep. 2018; 8(1):3029). The vaccine is then first diluted in an Eppendorf with culture medium to a 100-fold dilution. 100 μL of this dilution is then transferred to the wells of the plate containing the cells. The final dilution of the vaccine is 1000-fold. This dilution was determined to be the dilution with the best response in the dilution range evaluated in the pre-test. 500 μL of different serum dilution are then added in each well, dilution range from 1/4 to 1/1024. Negative and positive controls were carried out. The negative controls consisted of 400 μL of cell suspension, 100 μL of culture medium (DMEM) and 500 μL of VAXZEVRIA vaccinated serum-sample of respondent subjects. The positive controls consisted of 400 μL of cell suspension, 100 μL of vaccine dilution and 500 μL of VAXZEVRIA vaccinated serum-sample of respondent subjects. The plate is left to incubate for 7 days at 37° C. and 5% CO2. The incubation time results from pre-testing and microscopic assessment of cell death.

Day 2: The supernatant in the plate is considered as the sample to be analyzed with the chosen ELISA kit.

The calibration curves and the intrapolation on the basis of the inhibition curves have been realized with the GraphPad prism software.

### Results

### Case Description

An 83-year-old woman presented at the emergency room with an alteration of her general condition. She presented with symptoms of weakness, nausea, vomiting, weight loss and spontaneous bruises without any obvious reason, 14 days after having received her first dose of ChadOx1 nCov-19. She did not receive heparin or derivative during the last four months. Clinical examination unraveled bruising on the upper limbs. Computer Tomography (CT) of thorax and abdomen was normal. Oxygen saturation was 98% at admission and the subject was tested negative for SARS-CoV-2 infection as assessed by reverse-transcriptase polymerase chain reaction (RT-PCR). The initial laboratory investigations on the day of admission revealed that the subject was suffering from marked thrombocytopenia (i.e., platelet count of 10,000 per mm3), dramatically increased D-dimers plasma levels (i.e., >20,000 ng/mL) and slightly low plasma fibrinogen (i.e., 179 mg/dL). She was transfused with a platelet concentrate (±3.5×1011 platelets) on the day of admission.

During the night, the subject developed a grade NYHA 4 dyspnea. Pulmonary ventilation and perfusion (V/Q) scan were performed and disclosed bilateral pulmonary embolism. Anti-PF4 IgG antibodies (i.e., 1.80 AU/mL) were detected on day 1 post-admission using a PF4/polyvinylsulfonate rapid assay (HemosIL® AcuStar HIT IgG assay, Instrumentation Laboratory Belgium NV, Zaventem, Belgium). The diagnosis of VITT was confirmed using a heparin-induced multi-electrode aggregometry method (Journal of Thrombosis and Haemostasis. 2016; 14(12):2548-52). In face of the clinical picture, i.e., thrombocytopenia and thrombosis, with the presence of anti-PF4 antibodies and positive platelet activation tests within 30 days after vaccination with ChadOx1 nCov-19, VITT was diagnosed (Robert T. Proposed Brighton Collaboration process for developing a standard case definition for study of new clinical syndrome X, as applied to Thrombosis with Thrombocytopenia Syndrome (TTS)—V10.16.3 2021). The subject therefore promptly received 15 grams of IVIg (Privigen®, CSL Behring Gmbh, Marburg, Germany) and methylprednisolone 1 mg/kg. A second platelet concentrate (±4.5×1011 platelets) was administered for allowing initiation of anticoagulation as platelet count was still below 30,000 per mm³ (Guidance produced from the Expert Haematology Panel (EHP) focussed on Covid-19 Vaccine induced Thrombosis and Thrombocytopenia (VITT) Updated Guidance on Management. Version 1.3 2021). Platelet count rapidly improved, i.e. 53,000 per mm³, and anticoagulation was started with fondaparinux 5 mg od subcutaneously from day 1 to day 3 (taking into account renal failure, i.e. Cockcroft-Gault creatinine clearance <50 mL/min). She received additional IVIg on day 2 and 3, at the dose of 60 grams per day. Fondaparinux dose was increased to 7.5 mg od from day 4 to day 11 since renal function improved. On day 6, the subject was stabilized, and her global health status was improved as witnessed by normalized platelet count, decrease in D-dimers (i.e., from >20,000 ng/mL at admission to 14,380 ng/mL) and CRP (from 145 mg/dL at admission to 23 mg/dL). Later that day, however, oxygen saturation dropped below 80%. Cough with sputum production was noted and exacerbation of COPD with Moraxella catarrhalis infection was diagnosed. Oxygen supplementation was then started (2 liters per minute) combined with oral moxifloxacin 400 mg od for 5 days. On day 12 post-admission, anticoagulation was switched from fondaparinux to apixaban 5 mg bid.

Unfortunately, the clinical status worsened on day 12 post-admission with a de novo reduction of platelet count. The last intake of apixaban was on day 13 in the morning and never resumed. Apixaban plasma level on day 14 in the morning was 353 ng/mL (usual range: 22 to 177 ng/mL) and was still 132 ng/mL on day 15 in the morning. She died later that day from cardiovascular collapse.

### Results

A pre-testing was performed to determine the vaccine dilution at which the manipulation would be performed (dilution giving the best signal in the calibration curve i.e., 1000-fold).

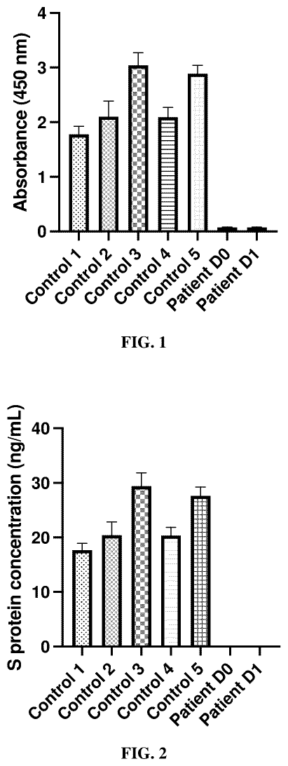

The results obtained for the standards at different concentrations allow to obtain a sigmoid calibration curve (Table 1). The results of the serum analysis of the subject not responding to VAXZEVRIA are given in Table 2. On the basis of the different absorbance values obtained for the dilutions of the subject's serum, it can easily be seen that, despite a dilution up to 1/1024, all values remain below the limit of quantification of the test. The results obtained with the VAXZEVRIA double-vaccinated subject serum samples are presented in Table 3. The comparison of the different values obtained for the control cases and for the subject's sera analyses are shown in FIGS. 1 and 2.

TABLE 1
Results obtained for the calibration curve.

| SARS-CoV-2 Spike S1 | | |
|---|---|---|
| (RBD Protein Standard | Replicat 1 | Replicat 2 |
| Concentration (ng/mL)) | Absorbance | Absorbance |
| 150.00 | 4.00 | 4.00 |
| 75.00 | 4.00 | 4.00 |
| 37.50 | 3.51 | 3.75 |
| 18.80 | 1.72 | 1.96 |
| 9.38 | 0.91 | 0.99 |
| 4.69 | 0.68 | 0.62 |
| 2.34 | 0.46 | 0.45 |
| 0.00 | 0.24 | 0.25 |

TABLE 2
Results obtained for the different dilutions
of the VAXZEVRIA non respondent serum sample.

| | Sample D0 | Sample D1 |
|---|---|---|
| Sample dilution factor | Absorbance | Absorbance |
| ¼ | 0.07 | 0.07 |
| ⅛ | 0.07 | 0.07 |
| 1/16 | 0.06 | 0.08 |
| 1/32 | 0.06 | 0.08 |
| 1/64 | 0.06 | 0.07 |
| 1/128 | 0.08 | 0.06 |
| 1/256 | 0.07 | 0.06 |
| 1/512 | 0.08 | 0.08 |
| 1/1024 | 0.07 | 0.07 |

TABLE 3
Results obtained with the VAXZEVRIA double-vaccinated subject serum samples.

| | VAXZEVRIA | VAXZEVRIA | VAXZEVRIA | VAXZEVRIA | VAXZEVRIA |
|---|---|---|---|---|---|
| | double- | double- | double- | double- | double- |
| | vaccinated | vaccinated | vaccinated | vaccinated | vaccinated |
| Sample | subject 1 | subject 2 | subject 3 | subject 4 | subject 5 |
| dilution | Concentration | Concentration | Concentration | Concentration | Concentration |
| factor | (ng/mL) | (ng/mL) | (ng/mL) | (ng/mL) | (ng/mL) |
| ¼ | 16.30 | 22.53 | 30.23 | 23.32 | 30.21 |
| ⅛ | 17.26 | 21.50 | 28.77 | 20.65 | 28.61 |
| 1/16 | 17.41 | 23.93 | 30.71 | 20.17 | 29.18 |
| 1/32 | 15.47 | 17.49 | 29.28 | 21.56 | 26.08 |
| 1/64 | 18.52 | 16.18 | 32.64 | 19.43 | 26.80 |
| 1/128 | 17.90 | 19.84 | 29.12 | 19.50 | 27.71 |
| 1/256 | 17.90 | 19.63 | 30.39 | 19.69 | 25.22 |
| 1/512 | 18.83 | 21.00 | 23.69 | 17.93 | 26.54 |
| 1/1024 | 19.49 | 21.72 | 29.90 | 20.76 | 28.43 |

### CONCLUSION

Based on the analyses performed and the results obtained, it can be assumed that the clinical case presented herein developed a form of immunity against the adenovirus used in the ChADOx1 nCoV-19 vaccine. Indeed, when the serum of the subject was put in contact with the vaccine cell model, an absence of response was observed, which is not the case with controls. The origin of this immunity is still unknown but this test allows to eliminate a possible lymphocytic or myolocytic origin. In the case of a failed vaccine response due to lymphocyte or bone marrow disorders, the test presented would have shown no inhibition of protein S production because the serum in this case would not have contained any anti-ChAdOx1 nCoV-19 antibodies.

1-15. (canceled)

16. An in vitro method for assessing the ability of a subject to produce at least one therapeutic molecule of interest encoded by a non-integrative viral vector, comprising the steps of:
(i) culturing cells expressing an attachment receptor specific of said non-integrating viral vector;
(ii) incubating said cells with a composition comprising said non-integrating viral vector and a serum sample of said subject in a culture medium;
(iii) measuring the expression level of said at least one therapeutic molecule of interest encoded by said non-integrative viral vector;
(iv) comparing the expression level measured in step (iii) with a reference value;
(v) assessing whether the subject is able to produce the at least one molecule of interest, wherein an expression level of the at least one molecule of interest superior to a reference value means that the subject is able to produce the at least one molecule of interest.

17. The in vitro method according to claim 16, wherein the ability to produce said at least one therapeutic molecule of interest means that the subject is not resistant towards said non-integrating virus vector.

18. The in vitro method according to claim 16, wherein said non-integrating virus vector is an adenovirus vector or an adeno-associated virus (AAV) vector.

19. The in vitro method according to claim 16, wherein the reference value is 0.

20. The in vitro method according to claim 16, wherein the reference value is the expression level of said at least one therapeutic molecule in the serum of a subject not incubated with the non-integrative viral vector encoding said at least one therapeutic molecule.

21. The in vitro method according to claim 16, wherein said at least one therapeutic molecule of interest is selected from the group comprising or consisting of viral protein Spike (S), Ebola virus glycoprotein (EBOV GP), Hemagglutinin (HA), Glycoprotein 41 and 120 (Gp41, Gp120), Falciparum circumsporozoite surface antigen, hepatitis C virus NS antigen, mycobacterial antigens 85A, 85B, and TB10.4, CHIKV full-length structural polyprotein (Capsid, E3, E2, 6k and E1), RSV-fusion protein, GI.1 and GII.4 antigen protein from norovirus, factor VIII, factor IX and factor XI.

22. The in vitro method according to claim 16, wherein said non-integrating virus vector is comprised in a vaccine composition or in a pharmaceutical composition.

23. The in vitro method according to claim 22, wherein said vaccine is for immunization against a pathogen selected from the group comprising or consisting of Coronaviridae, Filoviridae, Orthomyxoviridae, Togaviridae, Pneumoviridae, Caliciviridae, Retroviridae, Flaviviridae, Mycobacteriaceae, and Plasmodium.

24. The in vitro method according to claim 22, wherein said vaccine is for immunization against a virus selected from the group comprising or consisting of SARS-Cov-2, Ebola virus, Chikungunya virus, human immunodeficiency virus (HIV), hepatitis C virus (HCV), Zika virus, respiratory syncytial virus (RSV), Norovirus, Influenza virus.

25. The in vitro method according to claim 22, wherein said vaccine is for immunization against SARS-Cov-2.

26. The in vitro method according to claim 16, wherein said attachment receptor is selected from the group comprising or consisting of the coxsackievirus and adenovirus receptor (CAR), CD46, CD80, CD86, sialic acid and integrins.

27. The in vitro method according to claim 16, wherein said cells are CAR-expressing cells of a cell line selected from the group comprising or consisting of A549, H460, Caco-2, HEK-293.

28. The in vitro method according to claim 16, wherein said cells are CAR-expressing cells of a cell line A549.

29. The in vitro method according to claim 16, wherein step (ii) is performed during at least 5 days.

30. A kit for implementing the in vitro method according to claim 16, wherein said kit comprises means for determining or measuring said at least one therapeutic molecule of interest.

31. An in vitro method to assess the susceptibility of a subject to respond to a treatment with a non-integrative viral vector encoding at least one molecule of interest, comprising the steps of:
(i) culturing cells expressing an attachment receptor specific of the non-integrating viral vector;
(ii) incubating the cells with a composition comprising the non-integrating viral vector and a serum sample of the subject in a culture medium;
(iii) measuring the expression level of the at least one molecule of interest; and
(iv) assessing if the subject is susceptible to respond to the treatment, wherein an expression level of the at least one molecule of interest superior to a reference value means that the subject is susceptible to respond to the treatment.

32. A method for preventing and/or treating a disease with a non-integrative viral vector encoding at least one molecule of interest in a subject, comprising the steps of:
determining if the subject is susceptible to respond to a treatment with the non-integrative viral vector encoding the at least one molecule of interest by:
(i) culturing cells expressing an attachment receptor specific of the non-integrating viral vector;
(ii) incubating the cells with a composition comprising the non-integrating viral vector encoding the at least one molecule of interest, and a serum sample of the subject in a culture medium;
(iii) measuring the expression level of the at least one molecule of interest; and
(iv) determining if the subject is susceptible to respond to a treatment with the non-integrative viral vector, wherein an expression level of the at least one molecule of interest superior to a reference value means that the subject is susceptible to respond, and
administering to the subject susceptible to respond a therapeutically effective amount of the non-integrative viral vector encoding at least one molecule of interest.
